(19) United States
(12) Patent Application Publication
(10) Pub. No.: US 2026/0021220 A1
(43) Pub. Date: Jan. 22, 2026

(54) Hydrogel Film Composition and Method for Treating Skin Conditions

(71) Applicant: Takashi Yoshimura, La Jolla, CA (US)

(72) Inventor: Takashi Yoshimura, La Jolla, CA (US)

(21) Appl. No.: 18/778,742

(22) Filed: Jul. 19, 2024

(51) Int. Cl. A61L 15/44; A61L 15/20; A61L 15/28; A61L 15/32; A61L 15/40

(52) U.S. Cl. CPC A61L 15/44; A61L 15/20; A61L 15/28; A61L 15/325; A61L 15/40; A61L 2300/216; A61L 2300/236; A61L 2300/252; A61L 2300/30; A61L 2300/602

(57) ABSTRACT

The present invention relates to a pharmaceutical composition and method for treating skin conditions. The composition comprises a hydrogel film containing an effective amount of at least one active ingredient selected from hydrolyzed collagen, sodium hyaluronate, aloe barbadensis leaf extract, tocopheryl acetate, and rosa rugosa flower oil, along with one or more gelling agents capable of forming a co-polymer network. The hydrogel film is configured to adhere to the skin and deliver the active ingredient in a targeted and prolonged manner. The method involves applying the film to an affected area of the skin for a period sufficient to treat conditions such as sunburn, dryness, and eczema. The invention provides a convenient and effective means of delivering active ingredients directly to the skin while maintaining the structural integrity of the film during use.

### BACKGROUND

### Field of the Invention

The present invention relates generally to the field of dermatology and more specifically to hydrogel film compositions and methods for treating various skin conditions. In particular, the invention pertains to biocompatible hydrogel layers containing treatment agents and a backing layer for targeted delivery to affected skin areas on the face and body.

### Description of Related Art

Skin conditions such as sunburn, dryness, eczema, and other irritations are prevalent among a significant portion of the population. In fact, studies show that in any given year, more than one-third of Americans experience sunburn, nearly 30% suffer from dry skin, and 10-20% are affected by eczema. Current treatments often involve topical application of serums, moisturizers, or medications. However, these products tend to rub off or absorb into clothing and bedding, reducing their effectiveness.

Prior art, such as that disclosed in U.S. Patent Application Publication No. 2010/0055161 (hereinafter referred to as “the '161 publication”), has attempted to address these issues. The '161 publication discloses a device for delivering treatment agents to the skin, comprising a biocompatible hydrogel layer, one or more treatment agents included in the hydrogel layer, and a backing layer associated with the hydrogel layer. The treatment agents are configured for treating and/or preventing various skin conditions, and the hydrogel layer can be shaped and sized for application to different areas of the face and body.

While the '161 publication provides a foundation for targeted skin treatment using hydrogel compositions, there remains a need for an improved solution that offers prolonged delivery of active ingredients while maintaining structural integrity on the skin. The present invention aims to fulfill this need and overcome the limitations of conventional skincare products.

### SUMMARY

This summary is provided to introduce a selection of concepts, in a simplified format, that are further described in the detailed description of the invention. This summary is neither intended to identify key or essential inventive concepts of the invention nor is it intended for determining the scope of the invention.

The present invention addresses the need for an improved topical composition and method for treating various skin conditions, such as sunburn, dryness, and eczema. The invention provides a hydrogel film that can be applied directly to affected areas of the skin, delivering active ingredients in a targeted and prolonged manner.

In one aspect, the invention is directed to a pharmaceutical composition for topical application to skin, comprising a hydrogel film that includes an effective amount of at least one active ingredient selected from hydrolyzed collagen, sodium hyaluronate, aloe barbadensis leaf extract, tocopheryl acetate, and rosa rugosa flower oil. The hydrogel film also contains one or more gelling agents capable of forming a co-polymer network through hydrogen bonding, water, and optionally, one or more pharmaceutically acceptable excipients. The hydrogel film is configured to adhere to and deliver the active ingredient to the skin.

In various embodiments, the hydrogel film may further comprise a pH adjusting agent, a preservative, or a moisturizing agent. The film can be cut to a desired size and shape for application to a targeted area of the skin and is designed to remain adhered for a period of at least 4 hours. The hydrogel film may be translucent or transparent and can deliver the active ingredient to the skin at a controlled rate over time. For convenience, the hydrogel film can be packaged in a moisture-resistant foil pouch or as part of a kit comprising a plurality of individually packaged films.

In another aspect, the invention provides a method of treating skin conditions such as sunburn, dryness, and eczema. The method involves applying the hydrogel film to an affected area of the skin and allowing it to remain in contact with the skin for a period sufficient to deliver the active ingredient. The hydrogel film used in the method may have a thickness ranging from about 0.1 mm to about 2 mm and can be in the form of a sheet having dimensions of about 4 inches by 8 inches.

The present invention offers several advantages over prior art compositions and methods. The hydrogel film provides a convenient and effective means of delivering active ingredients directly to the skin, while the co-polymer network formed by the gelling agents ensures that the film maintains its structural integrity during use. The ability to cut the film to a desired size and shape allows for customized application to specific areas of the face or body. Furthermore, the prolonged adhesion of the film to the skin enables sustained delivery of the active ingredients, improving treatment outcomes.

In summary, the hydrogel film composition and method of the present invention represent a significant advancement in the field of dermatology, offering a targeted, convenient, and effective solution for treating a variety of common skin conditions.

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 illustrates a cross-sectional view of a hydrogel film composition for topical application to the skin.

FIG. 2 illustrates a perspective view of the hydrogel film 10 and its application to the skin, as well as its packaging.

FIG. 3 illustrates a flow chart depicting a method of using the hydrogel film kit for treating various skin conditions.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can,” “could,” “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

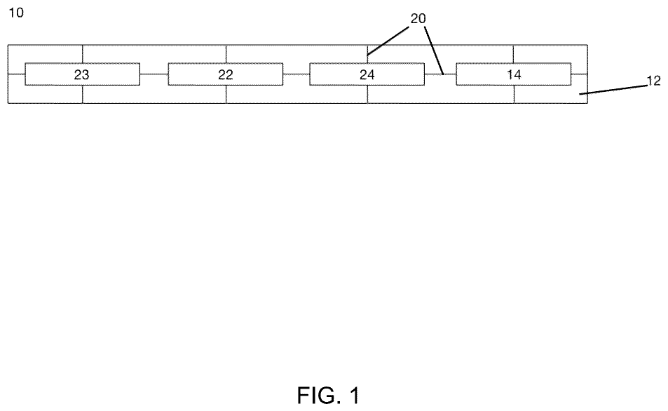

FIG. 1 illustrates a cross-sectional view of a hydrogel film composition 10 for topical application to the skin. The hydrogel film 10 comprises a hydrogel matrix 12 that includes an effective amount of at least one active ingredient 14 selected from the group consisting of hydrolyzed collagen, sodium hyaluronate, aloe barbadensis leaf extract, tocopheryl acetate, and rosa rugosa flower oil. The active ingredients 14 are homogeneously dispersed throughout the hydrogel matrix 12.

The hydrogel matrix 12 is formed by one or more gelling agents capable of creating a co-polymer network 20 through hydrogen bonding interactions. The gelling agents may be selected from carrageenan, gellan gum, pectin, alginate, and combinations thereof. The co-polymer network 20 provides structural integrity to the hydrogel film 10 and allows for controlled release of the active ingredients 14 upon application to the skin.

The hydrogel film 10 further comprises water as a solvent for the hydrogel matrix 12. Optionally, the hydrogel film 10 may include one or more pharmaceutically acceptable excipients 24, such as pH adjusting agents, preservatives, and moisturizing agents. The pH adjusting agents may be selected from citric acid, lactic acid, glycolic acid, hydrochloric acid, sodium hydroxide, potassium hydroxide, and combinations thereof. Suitable preservatives include benzalkonium chloride, sodium benzoate, potassium sorbate, phenoxyethanol, and combinations thereof. Moisturizing agents 26, such as glycerin, propylene glycol, butylene glycol, and combinations thereof, may also be incorporated into the hydrogel film 10.

The hydrogel film 10 is designed to adhere to the skin upon application, allowing for the delivery of the active ingredients 14 to the affected area. The hydrogel film 10 may be translucent or transparent, depending on the specific composition and concentrations of the components. The co-polymer network 20 enables the hydrogel film 10 to deliver the active ingredients 14 to the skin at a controlled rate over a prolonged period, providing sustained therapeutic benefits.

The hydrogel film composition 10 is prepared by mixing the gelling agents active ingredients 14, water 22, and optional pharmaceutically acceptable excipients 24 to form an aqueous solution. The solution is then cast onto a suitable substrate or mold and allowed to solidify, forming the hydrogel film 10 with the desired thickness and shape. The hydrogel film 10 may be cut or trimmed to the appropriate size for the intended application site on the skin.

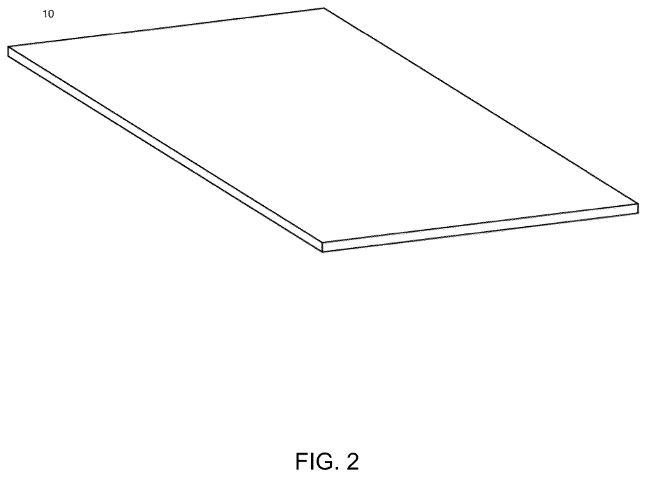

FIG. 2 illustrates a perspective view of the hydrogel film 10 and its application to the skin, as well as its packaging. The hydrogel film 10 comprises a hydrogel matrix 12 containing an effective amount of at least one active ingredient selected from the group consisting of hydrolyzed collagen, sodium hyaluronate, aloe barbadensis leaf extract, tocopheryl acetate, and rosa rugosa flower oil. The hydrogel matrix 12 further includes one or more gelling agents capable of forming a co-polymer network 20 through hydrogen bonding, water, and optionally, one or more pharmaceutically acceptable excipients 24.

As shown in FIG. 2, the hydrogel film 10 is configured to be cut to a desired size and shape for targeted application to an affected area of the skin. The hydrogel film 10 has a thickness ranging from about 0.1 mm to about 2 mm, which allows for easy cutting and shaping to fit the contours of the skin. In one embodiment, the hydrogel film 10 is in the form of a sheet having a length of approximately 4 inches and a width of about 8 inches, providing a suitable size for application to various parts of the body.

The hydrogel film 10 is designed to adhere to the skin upon application, conforming to the skin surface and maintaining contact for a prolonged period. The film's adherence properties are imparted by the specific composition of the hydrogel matrix 12 and the co-polymer network formed by the gelling agents. Once applied, the hydrogel film 10 is configured to remain adhered to the skin for a period of at least 4 hours, ensuring sufficient time for the active ingredients to be delivered to the skin.

In some embodiments the hydrogel film 10 is translucent or transparent in appearance. This property allows for discreet application and monitoring of the skin beneath the film. The transparency of the film is achieved through the careful selection and balance of ingredients in the hydrogel matrix 12.

To maintain the integrity and effectiveness of the hydrogel film 10 prior to use, the film is packaged in a moisture-resistant foil pouch. The foil pouch protects the hydrogel film 10 from environmental factors such as humidity, light, and contaminants, preserving the film's composition and ensuring optimal performance upon application. In one embodiment, the hydrogel film 10 is individually packaged in a moisture-impermeable foil bag, allowing for convenient single-use applications.

The application of the hydrogel film 10 can be for the treatment of skin conditions such as sunburn, dryness, and eczema. The method involves removing the hydrogel film 10 from its packaging, cutting it to the desired size and shape, and applying it to the affected area of the skin. The hydrogel film 10 is then allowed to remain in contact with the skin for a period sufficient to deliver the active ingredients, providing targeted and prolonged relief from the skin condition.

A hydrogel film can be applied to a non-woven fabric. A non-woven fabric.

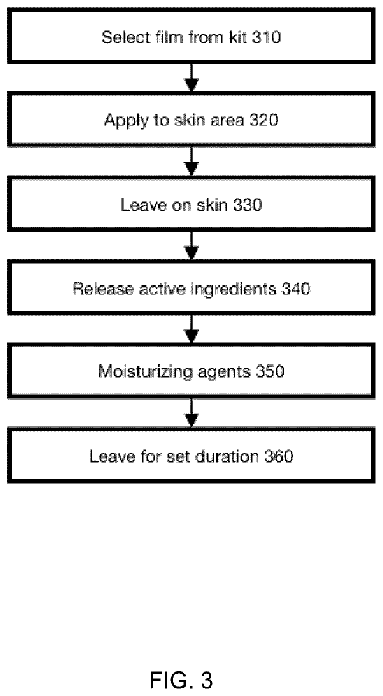

FIG. 3 illustrates a flow chart depicting a method of using the hydrogel film kit 32 for treating various skin conditions. The kit 32 comprises a plurality of individually packaged hydrogel films 10, each film having dimensions of approximately 4 inches in length and about 8 inches in width, as shown in the perspective view. The hydrogel films 10 are individually sealed within moisture-impermeable foil bags 30 to maintain their structural integrity and prevent dehydration during storage.

To use the hydrogel film kit 32 for treating skin conditions such as sunburn, dryness, or eczema, the user first selects an appropriately sized hydrogel film 10 from the kit 32 based on the dimensions of the affected skin area 34,310. The user then removes the hydrogel film 10 from its individual moisture-impermeable foil bag 30 and applies it directly to the affected skin area 34, ensuring that the entire area is covered by the film 320.

The hydrogel film 10 adheres to the skin surface, forming an occlusive barrier that prevents moisture loss and creates a favorable environment for the delivery of the active ingredients 14, 330. The co-polymer network 20 formed by the gelling agents within the hydrogel matrix 12 provides a controlled release of the active ingredients 14 over the treatment period, allowing for sustained therapeutic effects 340. The presence of moisturizing agents 26 and pH adjusting agents in the hydrogel film 10 further enhances the skin's hydration and maintains a suitable pH level for optimal skin healing and comfort 350.

The hydrogel film 10 is allowed to remain in contact with the skin 34 for a period sufficient to deliver the active ingredients 14 to the skin 34, typically ranging from about 1 hour to about 12 hours, depending on the severity of the skin condition and the desired treatment outcome 360.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A pharmaceutical composition for topical application to skin, comprising:
a hydrogel film comprising:
an effective amount of at least one active ingredient selected from the group consisting of hydrolyzed collagen, sodium hyaluronate, aloe barbadensis leaf extract, tocopheryl acetate, and rosa rugosa flower oil;
one or more gelling agents capable of forming a co-polymer network through hydrogen bonding;
water; and
optionally, one or more pharmaceutically acceptable excipients, wherein the hydrogel film is configured to adhere to and deliver the active ingredient to the skin.

2. The pharmaceutical composition of claim 1, wherein the one or more gelling agents are selected from the group consisting of carrageenan, gellan gum, pectin, alginate, and combinations thereof.

3. The pharmaceutical composition of claim 1, wherein the hydrogel film further comprises a pH adjusting agent selected from the group consisting of citric acid, lactic acid, glycolic acid, hydrochloric acid, sodium hydroxide, potassium hydroxide, and combinations thereof.

4. The pharmaceutical composition of claim 1, wherein the hydrogel film further comprises a preservative selected from the group consisting of benzalkonium chloride, sodium benzoate, potassium sorbate, phenoxyethanol, and combinations thereof.

5. The pharmaceutical composition of claim 1, wherein the hydrogel film is configured to be cut to a desired size and shape for application to a targeted area of the skin.

6. The pharmaceutical composition of claim 1, wherein the hydrogel film is configured to remain adhered to the skin for a period of at least 4 hours.

7. The pharmaceutical composition of claim 1, wherein the hydrogel film is translucent or transparent.

8. The pharmaceutical composition of claim 1, wherein the hydrogel film is configured to deliver the at least one active ingredient to the skin at a controlled rate over a period of time.

9. The pharmaceutical composition of claim 1, wherein the hydrogel film is packaged in a moisture-resistant foil pouch.

10. The pharmaceutical composition of claim 1, wherein the hydrogel film is part of a kit comprising a plurality of individually packaged hydrogel films, each film having dimensions of about 4 inches by 8 inches.

11. A pharmaceutical composition for topical application to skin, comprising:
a non-woven fabric comprising:
an effective amount of at least one active ingredient selected from the group consisting of hydrolyzed collagen, sodium hyaluronate, aloe barbadensis leaf extract, tocopheryl acetate, and rosa rugosa flower oil;
one or more gelling agents capable of forming a co-polymer network through hydrogen bonding;
water; and
optionally, one or more pharmaceutically acceptable excipients, wherein the non-woven fabric is configured to adhere to and deliver the active ingredient to the skin.

12. The pharmaceutical composition of claim 11, further comprising a hydrogel film positioned on the non-woven fabric.

13. A method of treating skin conditions selected from the group consisting of sunburn, dryness, and eczema, comprising:
applying one or more of a non-woven fabric and a hydrogel film to an affected area of the skin, comprising:
an effective amount of at least one active ingredient selected from the group consisting of hydrolyzed collagen, sodium hyaluronate, aloe barbadensis leaf extract, tocopheryl acetate, and rosa rugosa flower oil;
one or more gelling agents capable of forming a co-polymer network through hydrogen bonding;
water; and
optionally, one or more pharmaceutically acceptable excipients;
allowing the hydrogel film to remain in contact with the skin for a period sufficient to deliver the active ingredient to the skin.

14. The method of claim 11, wherein the one or more gelling agents are selected from the group consisting of carrageenan, gellan gum, pectin, alginate, and combinations thereof.

15. The method of claim 11, wherein the hydrogel film further comprises at least one moisturizing agent selected from the group consisting of glycerin, propylene glycol, butylene glycol, and combinations thereof.

16. The method of claim 11, wherein the hydrogel film further comprises at least one pH adjusting agent selected from the group consisting of citric acid, sodium hydroxide, potassium hydroxide, and combinations thereof.

17. The method of claim 11, wherein the hydrogel film is translucent or transparent.

18. The method of claim 11, wherein the hydrogel film has a thickness ranging from about 0.1 mm to about 2 mm.

19. The method of claim 11, wherein the hydrogel film is in the form of a sheet having a length of about 4 inches and a width of about 8 inches.

20. The method of claim 11, wherein the hydrogel film is individually packaged in a moisture-impermeable foil bag.
